SEQUENCE LISTING (24 sequences)

SEQ ID NO: 1 - protein, 800 aa, synthetic construct
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 2 - protein, 10 aa, synthetic construct
AKPSYPPTYK

SEQ ID NO: 3 - protein, 120 aa, synthetic construct
AKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYKAKPSYPPTYK

SEQ ID NO: 4 - protein, 472 aa, synthetic construct
LFSFFLLLTCTQLCLGTNRPDYNDDEEDDYKPPVYKPSPSKYRPVNPCLKKPCKYNGVCKPRGGSYKCFCKGGYYGYNCNLKNACKPNQCKNKSRCVPVGKTFKCVCRNGNFGRLCEKNVCSPNPCKNNGKCSPLGKTGYKCTCSGGYTGPRCEVHACKPNPCKNKGRCFPDGKTGYKCRCVDGYSGPTCQENACKPNPCSNGGTCSADKFGDYSCECRPGYFGPECERYVCAPNPCKNGGICSSDGSGGYRCRCKGGYSGPTCKVNVCKPTPCKNSGRCVNKGSSYNCICKGGYSGPTCGENVCKPNPCQNRGRCYPDNSDDGFKCRCVGGYKGPTCEDKPNPCNTKPCKNGGKCNYNGKIYTCKCAYGWRGRHCTDKAYKPNPCVVSKPCKNRGKCIWNGKAYRCKCAYGYGGRHCTKKSYKKNPCASRPCKNRGKCTDKGNGYVCKCARGYSGRYCSLKSPPSYDDDEY

SEQ ID NO: 5 - protein, 50 aa, synthetic construct
PWADYYGPKYGPPRRYGGGNYNRYGRRYGGYKGWNNGWKRGRWGRKYYGS

SEQ ID NO: 6 - protein, 750 aa, synthetic construct
YGRRYGEPSGYANIGHRRYYERAISFHRHSHVHGHHLLHRHVHRHSVLHGHVHMHRVSHRIMHRHRVLHGHVHRHRVLHNHVHRHSVLHGHVHRHRVLHRHVHRHNVLHGHVHRHRVLHKHVHNHRVLHKHLHKHQVLHGHVHRHQVLHKHVHNHRVLHKHLHKHQVLHGHVHTHRVLHKHVHKHRVLHKHLHKHQVLHGHIHTHRVLHKHLHKHQVLHGHVHTHRVLHKHVHKHRVLHKHLHKHQVLHGHVHMHRVLHKHVHKHRVLHKHVHKHHVVHKHVHSHRVLHKHVHKHRVEHQHVHKHHVLHRHVHSHHVVHSHVHKHRVVHSHVHKHNVVHSHVHRHQILHRHVHRHQVVHRHVHRHLIAHRHIHSHQAAVHRHVHTHFEGNFNDDGTDVNLRIRHGIIYFGGNTYRLSGGRRRFMTLWQECLESYGDSDECFVQLLEGNQHLFTVVQGHHSTSFRSDLSNDLHPDNNIEQIANDHVNDIAQSTDGDINDFADTHYNDVAPIADVHVDNIAQTADNHVKNIAQTAHHHVNDVAQIADDHVNDIGQTAYDHVNNIGQTADDHVNDIAQTADDHVNAIAQTADDHVNAIAQTADDHVNDIGDTANSHIVRVQGVAKNHLYGINKAIGKHIQHLKDVSNRHIEKLNNHATKNLLQSALQHKQQTIEREIQHKRHLSEKEDINLQHENAMKSKVSYDGPVFNEKVSVVSNQGSYNEKVPVLSNGGGYNGKVSALSDQGSYNEGYAY

SEQ ID NO: 7 - protein, 82 aa, synthetic construct
KHHHHHHSSEEYKGGYYPGNTYHYHSGGSYHGSGYHGGYKGKYYGKAKKYYYKYKNSGKYKYLKKARKYHRKGYKKYYGGSS

SEQ ID NO: 8 - protein, 103 aa, synthetic construct
IAALCGIVKSIDSDDSDYDYKGRGYCTNKGCRSGYNYFGNKGYCKYGEKSYTYNCNSYAGCCLPRNPYGKLKYYCTNKYGCPNNYYFYNNKGYYYLEHHHHHH

SEQ ID NO: 9 - protein, 199 aa, synthetic construct
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYKL

SEQ ID NO: 10 - protein, 171 aa, synthetic construct
AKPSYPPTYKAKPSYPPTYKAKPSYPPTYKAKPSYPPTYKAKPSYPPTYKAKPSYPPTYKPWADYYGPKYGPPRRYGGGNYNRYGRRYGGYKGWNNGWKRGRWGRKYYGSAKPSYPPTYKAKPSYPPTYKAKPSYPPTYKAKPSYPPTYKAKPSYPPTYKAKPSYPPTYKL

SEQ ID NO: 11 - protein, 175 aa, synthetic construct
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 12 - protein, 187 aa, synthetic construct
AKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGSSGSADYYGPKYGPPRRYGGGNYNRYGRRYGGYKGWNNGWKRGRWGRKYYGS

SEQ ID NO: 13 - protein, 187 aa, synthetic construct
PWADYYGPKYGPPRRYGGGNYNRYGRRYGGYKGWNNGWKRGRWGRKYYPWSSEEYKGGYYPGNTYHYHSGGSYHGSGYHGGYKGKYYGKAKKYYYKYKNSGKYKYLKKARKYHRKGYKKYYGGSSGSAKPSYPPTYKAKPSYPPTYKAKPSYPPTYKAKPSYPPTYKAKPSYPPTYKAKPSYPPTYK

SEQ ID NO: 14 - protein, 60 aa, synthetic construct
AKPSYPPTYKAKPSYPPTYKAKPSYPPTYKAKPSYPPTYKAKPSYPPTYKAKPSYPPTYK

SEQ ID NO: 15 - protein, 196 aa, synthetic construct
AKPSYPPTYKAKPSYPPTYKAKPSYPPTYKAKPSYPPTYKAKPSYPPTYKAKPSYPPTYKSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYK

SEQ ID NO: 16 - protein, 76 aa, synthetic construct
SSEEYKGGYYPGNTYHYHSGGSYHGSGYHGGYKGKYYGKAKKYYYKYKNSGKYKYLKKARKYHRKGYKKYYGGGSS

SEQ ID NO: 17 - protein, 5 aa, synthetic construct
GGGGS

SEQ ID NO: 18 - protein, 13 aa, synthetic construct
AKFVAAWTLKAAA

SEQ ID NO: 19 - protein, 218 aa, synthetic construct
MAKPSYPPTYKAKPSYPPTYKAKPSYPPTYKAKPSYPPTYKAKPSYPPTYKAKPSYPPTYKPWSSEEYKGGYYPGNTYHYHSGGSYHGSGYHGGYKGKYYGKAKKYYYKYKNSGKYKYLKKARKYHRKGYKKYYGGGSSAKPSYPPTYKAKPSYPPTYKAKPSYPPTYKAKPSYPPTYKAKPSYPPTYKAKPSYPPTYKLGGGGSAKFVAAWTLKAAA

SEQ ID NO: 20 - DNA, 654 bp, synthetic construct
  source 1..654: mol_type = other DNA
atggcgaaaccgagctatccgccgacctataaagcaaagccgtcttatccaccgacctacaaggcgaaaccaagctatccaccaacctataaggcgaaaccatcttatccgccaacctacaaagccaagccaagctacccgccaacatataaagccaaaccgtcttacccgccgacatacaaaccatggagctctgaagaatataaaggcggctattatccgggcaacacctaccattatcattctggcggcagctatcatggctctggctatcatggcggctataaaggcaaatattatggcaaagcgaaaaaatattattataaatataaaaacagcggcaaatataaatatctgaaaaaagcgagaaaatatcatagaaaaggctataaaaaatattatggcggcggcagctccgccaaaccttcttacccaccgacatataaggccaagccgagctacccaccaacatacaaggcaaaaccttcctatccacctacgtataaagcgaaacctagctatcctccgacgtacaaagcgaagccgtcctatccgcctacgtataaggcgaagccttcttatcctccaacgtacaagcttggcggtggcggtagcgcgaaatttgtggcggcctggaccctgaaggccgcggca

SEQ ID NO: 21 - protein, 14 aa, synthetic construct
QYIKANSKFIGITE

SEQ ID NO: 22 - protein, 15 aa, synthetic construct
ALNNRFQIKGVELKS

SEQ ID NO: 23 - protein, 16 aa, synthetic construct
GALNNRFQIKGVELKS

SEQ ID NO: 24 - protein, 23 aa, synthetic construct
SLLTEVETPIRNEWGCRCNDSSD

(19) United States
(12) Patent Application Publication
(10) Pub. No.: US 2026/0167681 A1
(43) Pub. Date: Jun. 18, 2026

(54) VACCINE DELIVERY CARRIER BASED ON ADHESIVE ADJUVANT PROTEIN AND ITS USES

(71) Applicant: POSTECH Research and Business Development Foundation, Pohang-si (KR)

(72) Inventors: Hyung Joon CHA, Pohang-si (KR); Sukwon JUNG, Pohang-si (KR); Hyun Tack WOO, Pohang-si (KR)

(73) Assignee: POSTECH Research and Business Development Foundation, Pohang-si (KR)

(21) Appl. No.: 19/180,607

(22) Filed: Apr. 16, 2025

(30) Foreign Application Priority Data
Apr. 16, 2024 (KR) 10-2024-0050944
Apr. 11, 2025 (KR) 10-2025-0047561

(51) Int. Cl. C07K 14/435; A61K 39/00; A61K 39/39; C07K 14/47

(52) U.S. Cl. CPC C07K 14/43504; A61K 39/39; C07K 14/47; A61K 2039/55516; A61K 2039/55544; A61K 2039/55555; A61K 2039/575; C07K 2319/00

(57) ABSTRACT

The present invention relates to a vaccine delivery carrier based on an adhesive adjuvant protein and its uses. The adhesive adjuvant protein according to the present invention exhibits excellent biocompatibility and outstanding immune-enhancing effects, making it applicable not only as a vaccine delivery carrier for carrying and delivering target antigens but also as an adjuvant for the treatment and research of various diseases.

### CROSS-REFERENCE TO RELATED PATENT APPLICATION

This application claims the benefit of Korean Patent Application No. 10-2024-0050944, filed on Apr. 16, 2024, and Korean Patent Application No. 10-2025-0047561, filed on Apr. 11, 2025, with the Korean Intellectual Property Office, the disclosure of which is incorporated herein in its entirety by reference.

### INCORPORATION BY REFERENCE OF SEQUENCE LISTING

The content of the electronically submitted sequence listing, file name: Q308372_sequence listing as filed; size: 26,069 bytes; and date of creation: Apr. 16, 2025, filed herewith, is incorporated herein by reference in its entirety.

### BACKGROUND OF THE INVENTION

### 1. Field of the Invention

The present invention relates to a vaccine delivery carrier based on an adhesive adjuvant protein and its uses.

### 2. Description of the Related Art

A vaccine is a pharmaceutical product that induces acquired immunity against a disease by introducing an antigen, which is either a component of a pathogen or an attenuated pathogen, into the human body to stimulate antibody production. Antigens developed to date include protein fragments derived from processed pathogens, attenuated viruses, and nucleic acids capable of triggering an immune response. Commercially available vaccines incorporate a vaccine delivery carrier that ensures the stable delivery of such antigens and/or an adjuvant that enhances immune cell activation to maximize vaccine efficacy.

These vaccine delivery carriers and adjuvants have been extensively researched, especially in response to the COVID-19 pandemic, which has heightened the demand for vaccine efficacy and durability. However, currently developed vaccine adjuvants and delivery carriers offer a very limited range of options and exhibit strong cytotoxicity, leading to severe inflammatory responses and compromised safety. Moreover, their low durability necessitates frequent booster vaccinations.

These vaccine delivery carriers and adjuvants have been extensively researched, especially in response to the COVID-19 pandemic, which has heightened the demand for vaccine efficacy and durability. However, currently developed adjuvants and vaccine delivery carriers offer a very limited range of options and exhibit strong cytotoxicity, leading to severe inflammatory responses and compromised safety. Moreover, their low durability necessitates frequent booster vaccinations.

To address these issues, extensive research has been conducted on peptide-based adjuvants and vaccine delivery carriers that offer greater efficiency and directly enhance immune responses with lower cytotoxicity compared to existing commercially available adjuvants and vaccine delivery carriers. However, peptide-based vaccine delivery carriers developed so far suffer from limitations due to their inherently low molecular weight, resulting in rapid in vivo clearance, reduced durability, and challenges in preserving antigens for effective delivery. Therefore, there is a need for the development of adjuvants and vaccine delivery carriers that can overcome these limitations.

Meanwhile, mussel adhesive proteins, derived from the byssus of mussels, are attracting considerable attention as biomaterials due to their excellent adhesive properties and biocompatibility. In particular, mussel adhesive proteins do not provoke an immune response in the human body, allowing them to be processed into various formulations such as hydrogels, nanofibers, and nanoparticles for the treatment of various diseases.

However, to date, no functional proteins with both underwater adhesive properties and immune-enhancing functions have been reported, nor have there been reports on nanocomplexes based on such proteins. Furthermore, no attempts have been made to load vaccines onto these nanocomplexes for use as a delivery and immune-enhancing system.

### SUMMARY OF THE INVENTION

Under the circumstances described above, the inventors have made extensive research efforts to develop a delivery carrier capable of stably and effectively delivering vaccines into cells, serving as a novel adjuvant that induces a more specific and direct immune response, in order to achieve optimized and safe preventive effects for the vaccines. To this end, the inventors have created an adhesive adjuvant sequence (MAP-AP; MAP-based Adhesive adjuvant protein), a fusion protein in which an adjuvant sequence (Adjuvant Peptide) is linked to a mussel adhesive protein (MAP), and have produced a MAP-AP-based nanocomplex with the target substance bound to the MAP-AP. After injecting the nanocomplex into the target subject, the inventors produced a nanovaccine through photoirradiation to evaluate its effective antigen delivery capability and immune-enhancing effects, thereby completing the present invention.

Therefore, an object of the present invention is to provide a fusion protein in which an adjuvant sequence is fused to a mussel adhesive protein.

Another object of the present invention is to provide a vaccine adjuvant composition.

Still another object of the present invention is to provide a nanocomplex.

Yet another object of the present invention is to provide a vaccine delivery carrier.

Still yet another object of the present invention is to provide a vaccine composition.

A further object of the present invention is to provide a method of producing a nanocomplex.

Another further object of the present invention is to provide a method of producing a nanovaccine.

Still another further object of the present invention is to provide a nanovaccine.

Still another object of the present invention is to provide a method for inducing an immune response in a subject, the method comprising administering the nanocomplex and a pharmaceutically acceptable carrier to the subject.

The terms used herein are for illustrative purposes only and should not be construed as limiting. Singular terms are intended to include the plural, unless the context clearly indicates otherwise. In the present disclosure, it should be understood that the terms such as “comprising,” “including,” or the like are intended to specify the presence of certain features, numbers, steps, operations, components, parts, or combinations thereof, and do not exclude the possibility of the presence or addition of one or more other features, numbers, steps, operations, components, parts, or combinations thereof.

Unless otherwise defined, all terms used herein, including technical or scientific terms, have the same meaning as commonly understood by one of ordinary skill in the art to which the embodiments pertain. Terms such as those defined in commonly used dictionaries should be interpreted to have meanings consistent with the context of the related art, and are not to be interpreted in an ideal or excessively formal meaning unless explicitly defined herein.

Hereinafter, the present invention will be described in more detail.

According to an aspect of the present invention, the present invention provides a fusion protein in which an adjuvant sequence is fused to a mussel adhesive protein.

Moreover, the present invention provides a nanocomplex and a nanovaccine based on a fusion protein in which an adjuvant sequence is fused to the mussel adhesive protein.

More specifically, the present invention provides a recombinant fusion protein, i.e., an adhesive adjuvant protein, which can specifically activate immune cells and provide an excellent immune response by adding at least one adjuvant sequence to the N-terminus or C-terminus of mussel adhesive protein with underwater adhesive properties. Furthermore, the present invention provides a MAP-AP-based nanocomplex bound to an antigen, which can enhance immunotherapeutic effects and secondary immune functions as a vaccine delivery carrier and an adjuvant.

The term “adjuvant” can be used interchangeably with the terms “immune booster,” “immune adjuvant,” or “immune-boosting adjuvant” and may refer to an immune adjuvant used when a vaccine antigen alone is insufficient to elicit an adequate immune response.

The term “immune boosting” may refer to inducing an initial immune response or enhancing an existing immune response to an antigen to a measurable level.

According to one embodiment of the present invention, the adhesive adjuvant protein can form spherical particles of several hundred nanometers in size due to electrostatic attraction with the antigen, thereby functioning as a vaccine delivery carrier with in vivo persistence and antigen retention. The present invention provides a method of manufacturing a nanovaccine as a vaccine delivery carrier containing an adhesive adjuvant protein, the method comprising producing a protein by genetically engineering a hydrophilic bio-derived protein with a functional sequence that has immune-enhancing effects, forming a nanocomplex through the interaction between an antigen and the protein, and reacting the nanocomplex with a photocrosslinkable compound. Compared to conventional adjuvants, which have raised concerns due to cytotoxicity from chemical substances, the present invention offers a safer and more sustainable alternative, reducing the need for frequent booster vaccinations and ensuring a more stable vaccine supply.

In the present invention, the term “mussel adhesive protein” refers to an adhesive protein derived from mussels, preferably including a mussel adhesive protein or a mutant thereof derived from Mytilus edulis, Mytilus galloprovincialis, or Mytilus coruscus, but is not limited thereto.

For example, the mussel adhesive protein of the present invention may include Mytilus edulis foot protein (Mefp)-1, Mytilus galloprovincialis foot protein (Mgfp)-1, Mytilus coruscus foot protein (Mcfp)-1, Mefp-2, Mefp-3, Mgfp-3, and Mgfp-5, or mutants thereof, derived from the aforementioned mussel species, preferably including a protein selected from the group consisting of foot protein (fp)-1 (SEQ ID NO: 1), fp-1 mutant (SEQ ID NO: 2), fp-1 mutant (SEQ ID NO: 3), fp-2 (SEQ ID NO: 4), fp-3 (SEQ ID NO: 5), fp-4 (SEQ ID NO: 6), fp-5 (SEQ ID NO: 7), and fp-6 (SEQ ID NO: 8); a fusion protein in which one or more proteins selected from the above group are linked; or a mutant thereof, but is not limited thereto.

Moreover, the mussel adhesive protein of the present invention includes all mussel adhesive proteins disclosed in International Publication Nos. WO2006/107183 and WO2005/092920. Preferably, the mussel adhesive protein may include fusion proteins such as fp-151 (SEQ ID NO: 9), fp-131 (SEQ ID NO: 10), fp-353 (SEQ ID NO: 11), fp-153 (SEQ ID NO: 12), and fp-351 (SEQ ID NO: 13), but is not limited thereto.

Furthermore, the mussel adhesive protein of the present invention may include a polypeptide in which a decapeptide (SEQ ID NO: 2), which is repeated approximately 80 times in fp-1, is continuously linked from 1 to 12 times or more. Preferably, it may be an fp-1 mutant polypeptide (SEQ ID NO: 3) in which the decapeptide of SEQ ID NO: 2 is continuously linked 12 times, but is not limited thereto.

In addition, the mussel adhesive protein of the present invention may be a mutant of fp-151 (SEQ ID NO: 15), but is not limited thereto. The protein sequence of SEQ ID NO: 15 is a sequence in which linker sequences and other elements are excluded compared to SEQ ID NO: 9. Specifically, it is a fusion protein sequence in which the sequence of Mgfp-5 represented by SEQ ID NO: 16 is fused between the fp-1 mutant sequence represented by SEQ ID NO: 14.

The mussel adhesive proteins of the present invention may also be modified to include conservative amino acid sequences that retain the properties of the mussel adhesive proteins mentioned above. In other words, amino acid sequences having 70% or greater, preferably 80% or greater, more preferably 90% or greater, i.e., 95%, 96%, 97%, 98%, 99% or greater sequence identity with the amino acid sequences of the aforementioned SEQ ID NOS, which exhibit substantially equivalent effects, may also be within the scope of the present invention.

The mussel adhesive protein may have tyrosine residues converted into catechol compounds, with 10 to 100% of the total tyrosine residues preferably converted into catechol compounds. In most mussel adhesive proteins, the proportion of tyrosine in the entire amino acid sequence may range from approximately 1% to 50%. Tyrosine in the mussel adhesive protein can be converted into the catechol compound dopamine (DOPA) through a hydration process, in which a hydroxyl group (OH) is added. However, mussel adhesive proteins produced in Escherichia coli do not undergo the conversion of tyrosine residues. Therefore, it is preferable to perform a modification reaction to convert tyrosine into DOPA through a separate enzymatic and chemical treatment method. Methods for modifying tyrosine residues into DOPA in mussel adhesive proteins can be carried out using methods known in the art and are not particularly limited.

The catechol compound refers to a compound containing a dihydroxy group, which imparts adhesive properties to the mussel adhesive protein through cross-linking. Specifically, it may include at least one compound selected from the group consisting of 3,4-dihydroxyphenylalanine (DOPA), DOPA o-quinone, 2,4,5-trihydroxyphenylalanine (TOPA), TOPA quinone, and derivatives thereof.

The mussel adhesive protein may comprise one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16.

The mutants of the mussel adhesive protein in the present invention may preferably include additional sequences at the carboxyl or amino terminus of the mussel adhesive protein or some amino acids substituted with other amino acids, provided that the adhesive properties of the mussel adhesive protein are maintained. Preferably, a polypeptide consisting of 3 to 25 amino acids containing RGD is linked to the carboxyl or amino terminus of the mussel adhesive protein, or 1% to 100% of the total tyrosine residues in the mussel adhesive protein, more preferably 5% to 100%, may be substituted with 3,4-dihydroxyphenyl-L-alanine (DOPA), but is not limited thereto.

The mussel adhesive protein can be mass-produced using genetic engineering methods by inserting it into a conventional vector designed for expressing external genes, but is not limited thereto. The vector can be appropriately selected or newly created based on the type and characteristics of the host cell used to produce the protein. The method of transforming the vector into a host cell vector and the method of producing a recombinant protein from the transformant can be easily carried out using conventional methods. The selection, preparation, and transformation of the vector, as well as the expression of recombinant proteins, can be readily performed by those skilled in the art to which the present invention pertains, and any modifications of the conventional methods also fall within the scope of the present invention.

The adjuvant sequence of the present invention may include any adjuvant sequences known in the art, as long as they can achieve the objects of the present invention. It is preferably a T-helper epitope, but is not limited thereto. For example, the T-helper epitope may comprise at least one selected from the group consisting of a Pan DR-binding epitope (PADRE) (SEQ ID NO: 18), a T-helper epitope of tetanus toxoid peptide (TTP) (SEQ ID NO: 21), a T-helper epitope of influenza virus hemagglutinin (SEQ ID NO: 22 or SEQ ID NO: 23), and an epitope of human influenza virus M2e (SEQ ID NO: 24). In one embodiment of the present invention, it may be PADRE, but is not limited thereto.

The T-helper epitope can be recognized by one or more different mammalian species. Therefore, the epitope is not considered to be limited to the immune system of the species in which it is recognized. For example, a rodent T-helper epitope can be recognized by the immune system of mice, rats, rabbits, guinea pigs, other rodents, humans, or dogs.

Moreover, according to another aspect of the present invention, the present invention provides a vaccine adjuvant composition comprising the adhesive adjuvant protein.

Furthermore, according to still another aspect of the present invention, the present invention provides a method of inducing an immune response in a subject, comprising the step of administering the vaccine adjuvant composition to the subject.

The term “vaccine adjuvant” as used herein refers to a pharmaceutical or immunological preparation administered for the purpose of enhancing the immune response to a vaccine.

The vaccine adjuvant composition provided in the present invention, when administered together with an antigen, can enhance the antibody titer and simultaneously boost both cellular and humoral immunity. In particular, since the vaccine adjuvant contains an adhesive immune-enhancing protein (MAP-AP) as an active ingredient based on the safe mussel adhesive protein, it can be safely used in vivo.

The vaccine adjuvant composition may further include pharmaceutically acceptable adjuvants, excipients, or diluents in addition to the carrier.

The term “pharmaceutically acceptable” as used herein refers to a composition that is physiologically acceptable and does not cause allergic reactions such as gastrointestinal discomfort, dizziness or similar reactions when administered to humans. Examples of such carriers, excipients, and diluents may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. The composition may further include fillers, anticoagulants, lubricants, wetting agents, flavorings, emulsifiers, and preservatives.

In addition to the vaccine adjuvant of the present invention, the composition may further include other commonly used vaccine adjuvants. Examples of such vaccine adjuvants may include aluminum hydroxide, aluminum phosphate, alum (potassium aluminum sulfate), MF59, virosomes, AS04 [a mixture of aluminum hydroxide and monophosphoryl lipid A (MPL)], AS03 [a mixture of DLα-tocopherol, squalene, and polysorbate 80 (as an emulsifier)], CpG, flagellin, Poly I:C, AS01, AS02, ISCOMs, and ISCOMMATRIX.

Additionally, the vaccine adjuvant composition may be formulated using methods known in the art to allow for rapid release, sustained release, or delayed release of the active ingredient upon administration to a mammal. The formulation may be in the form of a powder, granule, tablet, emulsion, syrup, aerosol, soft or hard gelatin capsule, sterile injectable solution, or sterile powder.

The vaccine adjuvant composition of the present invention can be administered via various routes, including oral and parenteral administration. For example, it may be administered as a suppository or via transdermal, intravenous, intraperitoneal, intramuscular, intralesional, intranasal, or intrathecal routes. Additionally, it can be administered using an implantable device for sustained, continuous, or repeated release. The frequency of administration may be once or multiple times per day within a desired range, and the duration of administration is not particularly limited.

The vaccine adjuvant composition of the present invention can be administered through general systemic administration or local administration, such as intramuscular or intravenous injection. The administration route of the vaccine adjuvant composition of the present invention can be any conventional route that allows it to reach the target tissue. Such administration routes may include parenteral routes, for example, intraperitoneal, intravenous, intramuscular, subcutaneous, and intrasynovial administrations, but are not limited thereto.

The vaccine adjuvant composition of the present invention can be formulated into a suitable form with commonly used pharmaceutically acceptable carriers. Examples of such carriers may include parenteral carriers such as water, suitable oils, saline, aqueous glucose, and glycols, and may also include stabilizers and preservatives. Suitable stabilizers may include antioxidants such as sodium bisulfite, sodium sulfite, or ascorbic acid. Suitable preservatives may include benzalkonium chloride, methyl- or propyl-paraben, and chlorobutanol.

The vaccine adjuvant composition of the present invention may, depending on the administration method or formulation appropriately include suspending agents, solubilizers, stabilizers, isotonic agents, preservatives, anti-adsorption agents, surfactants, diluents, excipients, pH adjusters, anesthetics, buffers, antioxidants, and the like, as needed.

When the vaccine adjuvant composition of the present invention is administered to a subject, the dosage may vary depending on various factors, including the subject's height, body surface area, age, sex, and general health condition, the specific compound administered, the time and route of administration, and other drugs administered concurrently. For example, the composition may be administered in an amount ranging from 100 ng/kg of body weight to 10 mg/kg of body weight. In another example, it may be administered in an amount ranging from 1 to 500 μg/kg of body weight, and in yet another example, it may be administered in an amount ranging from 5 to 50 μg/kg of body weight.

The subject includes humans and other animals. Typically, the subject is a human. In another embodiment, the subject may be a non-human animal, preferably a non-human mammal, which may be selected from the group consisting of rats, mice, guinea pigs, hamsters, rabbits, monkeys, gorillas, chimpanzees, dogs, cats, cattle, horses, pigs, sheep, and goats, but is not limited thereto.

The composition of the present invention is administered in a pharmaceutically effective amount.

Moreover, according to yet another aspect of the present invention, the present invention provides a nanocomplex comprising the adhesive adjuvant protein (MAP-AP) bound to an antigen.

The nanocomplex includes a photocrosslinker and may utilize any photocrosslinker known in the art, as long as it can achieve the objects of the present invention. The photocrosslinker may be a photoinitiator or a photocatalyst, preferably a photoinitiator, but is not limited thereto.

For example, the photoinitiator may include at least one selected from the group consisting of ruthenium (Ru(II)), palladium (Pd(II)), copper (Cu(II)), nickel (Ni(II)), manganese (Mn(II)), and iron (Fe(III)). In one embodiment of the present invention, ruthenium (Ru(II)) was used in the form of [Ru(II)bpy₃]Cl₂ (tris(bipyridine)ruthenium(II) chloride); however, the invention is not limited thereto.

The photocatalyst may include at least one selected from the group consisting of persulfate, periodate, perbromate, perchlorate, vitamin B12, pentaamminechlorocobalt(III), ammonium cerium(IV) nitrate, oxalic acid, and EDTA.

Furthermore, according to still yet another aspect of the present invention, the present invention provides a vaccine delivery carrier comprising the nanocomplex.

The vaccine delivery carrier according to the present invention, which includes a mussel adhesive protein with inherent underwater adhesive properties, can effectively deliver the vaccine by enhancing the retention time of the target vaccine at the target site.

In addition, according to a further aspect of the present invention, the present invention provides a vaccine composition comprising the nanocomplex and a pharmaceutically acceptable carrier.

The composition may further comprise an adjuvant.

The composition enhances both cellular and humoral immunity, thereby achieving the desired effect.

The subject includes humans and other animals. Typically, the subject is a human. In another embodiment, the subject may be a non-human animal, preferably a non-human mammal, which may be selected from the group consisting of rats, mice, guinea pigs, hamsters, rabbits, monkeys, gorillas, chimpanzees, dogs, cats, cattle, horses, pigs, sheep, and goats, but is not limited thereto.

The vaccine composition of the present invention is administered in a pharmaceutically effective amount.

The term “pharmaceutically effective amount” as used herein refers to an amount sufficient to treat a disease with a reasonable benefit/risk ratio applicable to medical treatment. The effective dosage level can be determined based on factors including the type and severity of the subject, age, sex, the activity of the drug, the sensitivity to the drug, the time and route of administration, excretion rate, treatment duration, other drugs administered concurrently, and other factors well known in the medical field. For example, the composition of the present invention may be administered at a dosage ranging from 0.1 mg/kg to 1 g/kg. In another example, it may be administered at a dosage ranging from 1 mg/kg to 500 mg/kg, with the dosage being appropriately adjusted according to the patient's age, sex, and condition.

In the present invention, the antigen may be any antigen known in the art as long as it can induce an immune response in vivo. In one embodiment of the present invention, ovalbumin was used; however, the invention is not limited thereto.

In addition, according to still another further aspect of the present invention, the present invention provides a method of producing a nanocomplex, comprising the step of binding an antigen to an adhesive adjuvant protein (MAP-AP), a method of producing a nanovaccine, comprising the step of irradiating the nanocomplex with visible light, and a nanovaccine produced according to the method of producing a nanovaccine.

The nanocomplex and nanovaccine of the present invention are effective as adjuvants and vaccines because they enhance specific immune responses when used in combination with a given antigen based on the adhesive adjuvant protein (MAP-AP).

Additionally, according to another further aspect of the present invention, the present invention provides a method for inducing an immune response in a subject, comprising administering the vaccine composition comprising the nanocomplex and a pharmaceutically acceptable carrier to the subject.

The method further comprising irradiating the nanocomplex with visible light, which induces cross-linking of the fusion protein.

Since the method of the present invention utilizes the adhesive adjuvant protein (MAP-AP) of the present invention, the common details between the two are omitted herein to avoid excessive complexity in the specification.

The adhesive adjuvant protein according to the present invention exhibits excellent biocompatibility and outstanding immune-enhancing effects, making it applicable not only as a vaccine delivery carrier for carrying and delivering target antigens but also as an adjuvant for the treatment and research of various diseases.

### BRIEF DESCRIPTION OF THE DRAWINGS

The above and other features and advantages of the present invention will become more apparent by describing in detail exemplary embodiments thereof with reference to the attached drawings in which:

FIG. 1 is an overall schematic diagram of the nanocomplex using the adhesive adjuvant protein (MAP-AP) of the present invention and the nanovaccine produced through its photocrosslinking;

FIG. 2 shows an adhesive adjuvant protein (MAP-AP) that contains the PADRE sequence as an adjuvant sequence in the mussel adhesive protein (MAP) according to an example of the present invention;

FIG. 3 shows the SDS PAGE results comparing the molecular weight of the adhesive adjuvant protein (MAP-AP) with that of the conventional mussel adhesive protein;

FIG. 4 shows the MALDI-TOF results confirming the molecular weight of the adhesive adjuvant protein (MAP-AP);

FIG. 5 shows the surface morphology and structure of the nanocomplex formed by mixing the model antigen ovalbumin with the adhesive adjuvant protein (MAP-AP) (Before), and the nanovaccine formed by adding a photocrosslinkable compound to the nanocomplex (After), as observed through scanning electron microscopy;

FIG. 6A shows the comparison of the surface charge and particle size of the nanocomplexes and nanovaccines;

FIG. 6B compares the UV-vis spectra before (black) and after (red) photocrosslinking, showing the change in the peak after crosslinking;

FIG. 6C compares the surface charge of the nanocomplexes formed by mixing the model antigen ovalbumin with the adhesive adjuvant protein in different ratios, with the surface charge measured through zeta-potential analysis;

FIG. 7A shows the surface morphology and structure of the nanocomplex before and after freeze-drying, as observed through scanning electron microscopy;

FIG. 7B shows the particle size and surface charge of the nanocomplexes and nanovaccines before and after freeze-drying;

FIG. 8 shows the in vitro release profile of the nanovaccine;

FIG. 9A shows the adhesion ability of the nanovaccines under fluid flow, confirmed in vitro using a quartz crystal microbalance (QCM) device;

FIG. 9B shows the tissue adhesion ability of the nanovaccines confirmed ex vivo through fluorescence microscopy;

FIG. 10 shows the cell viability of the nanocomplexes and nanovaccines, determined through a CCK-8 assay;

FIG. 11A shows the biocompatibility of the nanovaccines, assessed 24 hours after drug delivery through Live/Dead assay;

FIG. 11B shows the biocompatibility of the nanovaccines, assessed 72 hours after drug delivery through Live/Dead assay;

FIG. 12A shows the in vivo persistence of the nanovaccines after injection into mice;

FIG. 12B shows the in vivo persistence of the nanovaccines after injection into mice;

FIG. 13 shows the concentration of antibodies that can specifically bind to antigens and the concentration of cytokines secreted from immune cells isolated from the spleen 2 and 4 weeks after injection of the nanovaccines into mice;

FIG. 14A shows the analysis of activated immune cells through FACS, 4 weeks after injection of the nanovaccines into mice; and

FIG. 14B shows the quantitative comparison of activated immune cells, 4 weeks after injection of the nanovaccines into mice.

### DETAILED DESCRIPTION OF THE INVENTION

Hereinafter, the present invention will be described in detail with reference to the following examples. However, the following examples are provided for illustrative purposes only and do not limit the scope of the present invention.

### Example 1: Production of Adhesive Adjuvant Protein (MAP-AP)

The inventors of the present invention prepared an adhesive adjuvant protein (MAP-based Adjuvant Protein, MAP-AP) by adding an adjuvant sequence to mussel adhesive protein (MAP).

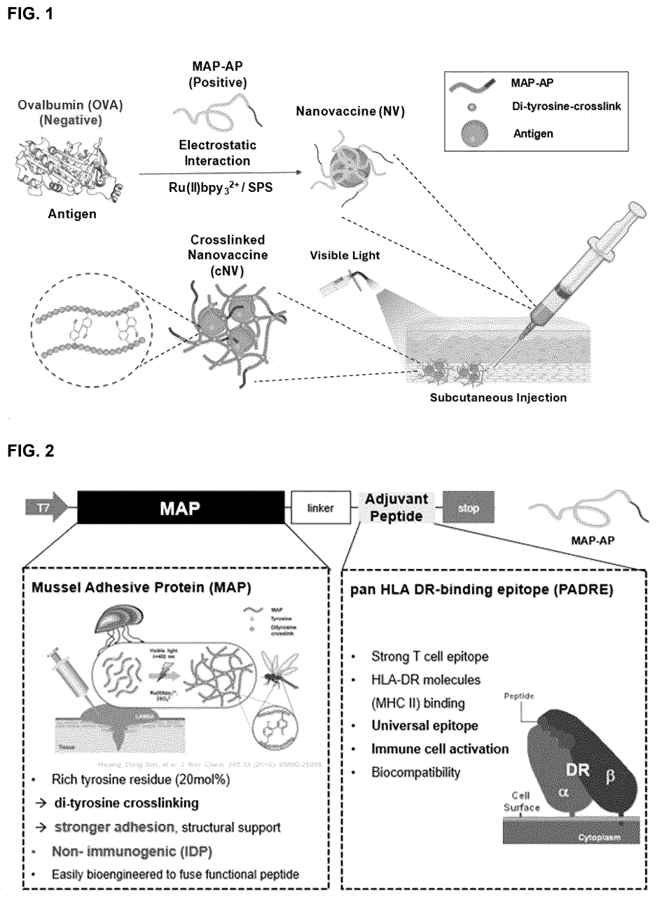

Specifically, to prepare the adhesive adjuvant protein (MAP-AP), a fusion protein (SEQ ID NO: 19) was designed in which the PADRE (pan HLA DR-binding epitope, SEQ ID NO: 18) as an adjuvant sequence was linked to the mussel adhesive protein (fp-151, SEQ ID NO: 9) via a linker (GGGGS, SEQ ID NO: 17) (FIG. 2). The amino acid sequence of the adhesive adjuvant protein (MAP-AP) and the nucleotide sequence encoding it are shown in Table 1.

TABLE 1

| | Sequence | SEQ ID NO. |
|---|---|---|
| MAP-AP | MAKPSYPPTYKAKPSYPPTYKAKPSYPPTYKAKPSYPPTYKAKPSYPPTYKAKPSYPPTYK | 19 |
| amino acid | PWSSEEYKGGYYPGNTYHYHSGGSYHGSGYHGGYKGKYYGKAKKYYYKYKNSGKYKYLKKA | |
| sequence | RKYHRKGYKKYYGGGSSAKPSYPPTYKAKPSYPPTYKAKPSYPPTYKAKPSYPPTYKAKPS | |
| | YPPTYKAKPSYPPTYKLGGGGSAKFVAAWTLKAAA | |
| MAP-AP | ATGGCGAAACCGAGCTATCCGCCGACCTATAAAGCAAAGCCGTCTTATCCACCGACCTACA | 20 |
| nucleotide | AGGCGAAACCAAGCTATCCACCAACCTATAAGGCGAAACCATCTTATCCGCCAACCTACAA | |
| sequence | AGCCAAGCCAAGCTACCCGCCAACATATAAAGCCAAACCGTCTTACCCGCCGACATACAAA | |
| | CCATGGAGCTCTGAAGAATATAAAGGCGGCTATTATCCGGGCAACACCTACCATTATCATT | |
| | CTGGCGGCAGCTATCATGGCTCTGGCTATCATGGCGGCTATAAAGGCAAATATTATGGCAA | |
| | AGCGAAAAAATATTATTATAAATATAAAAACAGCGGCAAATATAAATATCTGAAAAAAGCG | |
| | AGAAAATATCATAGAAAAGGCTATAAAAAATATTATGGCGGCGGCAGCTCCGCCAAACCTT | |
| | CTTACCCACCGACATATAAGGCCAAGCCGAGCTACCCACCAACATACAAGGCAAAACCTTC | |
| | CTATCCACCTACGTATAAAGCGAAACCTAGCTATCCTCCGACGTACAAAGCGAAGCCGTCC | |
| | TATCCGCCTACGTATAAGGCGAAGCCTTCTTATCCTCCAACGTACAAGCTTGGCGGTGGCG | |
| | GTAGCGCGAAATTTGTGGCGGCCTGGACCCTGAAGGCCGCGGCA | |

The plasmid containing the genetic information for the adhesive adjuvant protein (MAP-AP) was inserted into E. coli BL21 and cultured in LB medium (5 g/liter yeast extract, 10 g/liter tryptone, and 10 g/liter NaCl). When the absorbance of the culture reached approximately 0.7-0.8 at 600 nm, IPTG was added to a final concentration of 1 mM to induce the expression of MAP-AP. At this point, 10 ml of LB medium (with 500 μg of ampicillin added) was placed in a 50 ml sterilized tube, and the culture grown for 12 hours was inoculated into a 500 ml flask containing 100 ml of LB medium. The culture was then centrifuged at 4,000 rpm for 10 to 20 minutes to collect the cell pellet, which was subsequently stored at −80° C. The cell pellet was resuspended in 10-15 ml of lysis buffer (10 mM Tris-CI, 100 mM sodium phosphate, pH 8.0) per gram of cell pellet, and the cells were lysed at 20,000 PSI (Constant systems, Low March, UK). The cell lysate was then centrifuged at 18,000 g for 20 minutes at 4° C. to obtain the cell debris. The cell debris was diluted by adding 20 ml of acetic acid (5, 10, 15, 20, 25, 30, 22, 24, 26, and 28 (v/v) %) per gram, and centrifuged under the same conditions. The supernatant was freeze-dried and then stored.

### Example 2: Purity and Characterization of Adhesive Adjuvant Protein (MAP-AP)

An experiment was conducted to compare the molecular weight of the adhesive adjuvant protein (MAP-AP) produced in Example 1 with that of mussel adhesive protein.

Specifically, the control mussel adhesive protein (fp-151) and the adhesive adjuvant protein (MAP-AP) sample produced in Example 1 were diluted in 100 μl of SDS-PAGE buffer (0.5 M Tris-HCl (pH 6.8), 10% glycerol, 5% SDS, 5% β-mercaptoethanol, and 0.25% bromophenol blue) and denatured by boiling at 100° C. for 5 minutes. For SDS-PAGE, the samples were electrophoresed on a 15% SDS-polyacrylamide gel, and protein bands were detected using Coomassie blue staining.

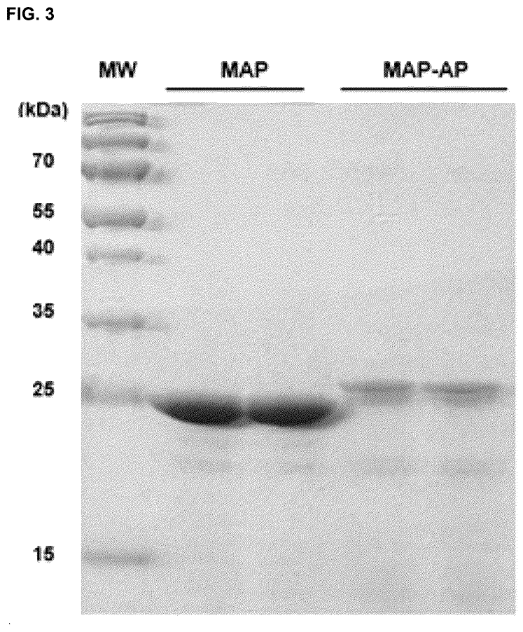

As a result, as shown in FIG. 3, the molecular weight of the adhesive adjuvant protein (MAP-AP) increased compared to the mussel adhesive protein.

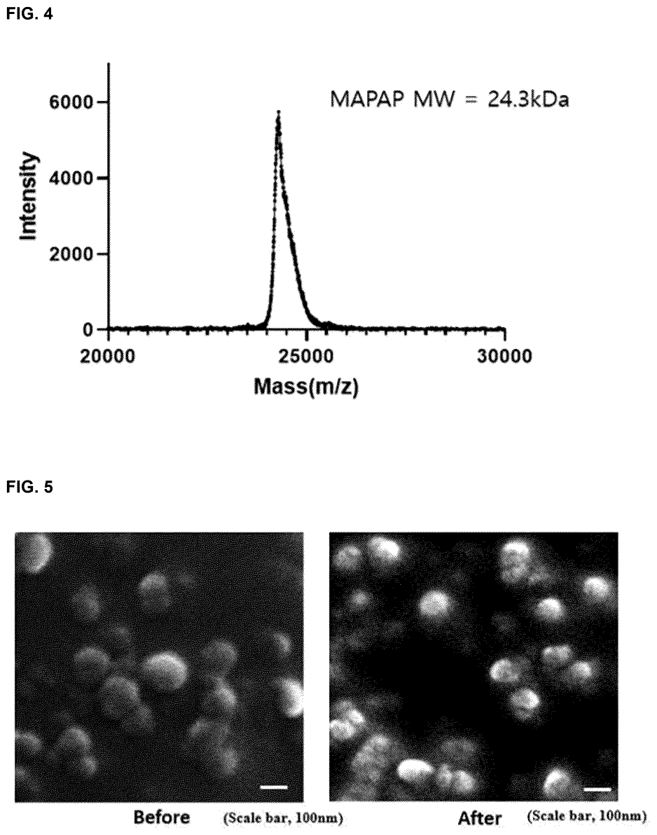

For a more precise measurement of the molecular weight, the free-dried adhesive adjuvant protein was analyzed using a matrix-assisted laser desorption/ionization time of flight (MALDI-TOF) mass spectrometer. As shown in FIG. 4, the molecular weight of the adhesive adjuvant protein determined to be 24.3 kDa, which was consistent with the value calculated from the amino acid sequence.

These results indicate that the adhesive adjuvant protein (MAP-AP) of the present invention was successfully produced.

### Example 3: Preparation of MAP-AP-Based Nanocomplex (NV) and Nanovaccine (cNV) Using the Same

The adhesive adjuvant protein (MAP-AP) produced in Example 1 was mixed with an antigen to prepare a nanocomplex, and a photocrosslinkable compound was added to the nanocomplex to produce a nanovaccine.

Specifically, to prepare the nanocomplex, ovalbumin (OVA), a representative antigen, was used. Solution A was prepared by mixing 200 μg of ovalbumin (1 mg/ml) with 10-50 mM sodium persulfate (SPS), and solution B was prepared by mixing 200 μg of MAP-AP (1 mg/ml) with 0.1-5 mM [Ru(II)bpy₃]Cl₂ (tris(bipyridine)ruthenium(II) chloride, hereinafter referred to as Rubpy) as a photocrosslinker (photoinitiator). Solutions A and B were mixed, and D.W. was added to prepare 1 ml of the MAP-AP solution. The resulting mixture was stirred in the dark for 30 minutes to obtain a stabilized MAP-AP-based nanocomplex (OVA/MAP-AP/Rubpy/SPS, hereinafter referred to as NV). Subsequently, the mixture was centrifuged at 9,000 rpm for 10 minutes to remove the supernatant, followed by resuspension and washing with 1 ml of D.W. After repeating the centrifugation and washing process twice, the nanocomplex (NV) was resuspended by adding a 1 mg/ml MAP-AP solution of the required volume based on the desired dose, followed by further centrifugation and washing. Alternatively, the obtained nanocomplex was freeze-dried and stored. Upon administration, a 1 w % MAP-AP solution of the required volume was added, and the nanocomplex was resuspended and administered accordingly. The nanovaccine (cNV) was formed by inducing in situ crosslinking through photoirradiation of the nanocomplex.

### Example 4: Characterization of MAP-AP-Based Nanocomplex (NV) and Nanovaccine (cNV)

The characteristics of the nanocomplex and nanovaccine using the adhesive adjuvant protein (MAP-AP) produced in Example 3 were analyzed.

4-1. Analysis of Surface Morphology and Structure of Nanocomplex (NV) and Nanovaccine (cNV)

First, the surface morphology and structure of the nanocomplex before photoirradiation and the nanovaccine after photoirradiation were observed.

Specifically, the nanocomplex before photoirradiation and the nanovaccine after photoirradiation were diluted in 10 ml of D.W., and 10 μl of each sample was placed on carbon tape. The samples were then freeze-dried for 12 hours. Subsequently, gold sputtering was performed on the nanocomplex and nanovaccine, and their particle morphology and structure were examined using a scanning electron microscope (SEM) at acceleration voltages of 5 kV and 20 kV.

As a result, as shown in FIG. 5, no significant changes in characteristics were observed before and after photoirradiation.

4-2. Analysis of Particle Size and Surface Charge of Nanocomplex (NV) and Nanovaccine (cNV)

Additionally, an experiment was conducted to compare the particle size and surface charge before and after photoirradiation.

Specifically, dynamic light scattering (DLS, Zetasizer, UK) was used to measure the particle size and surface charge. The particle concentration was maintained below 1 mg/mL, and all measurements were performed at room temperature.

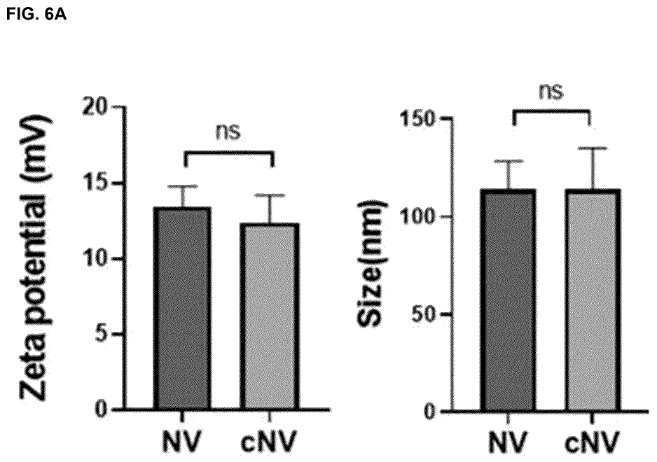

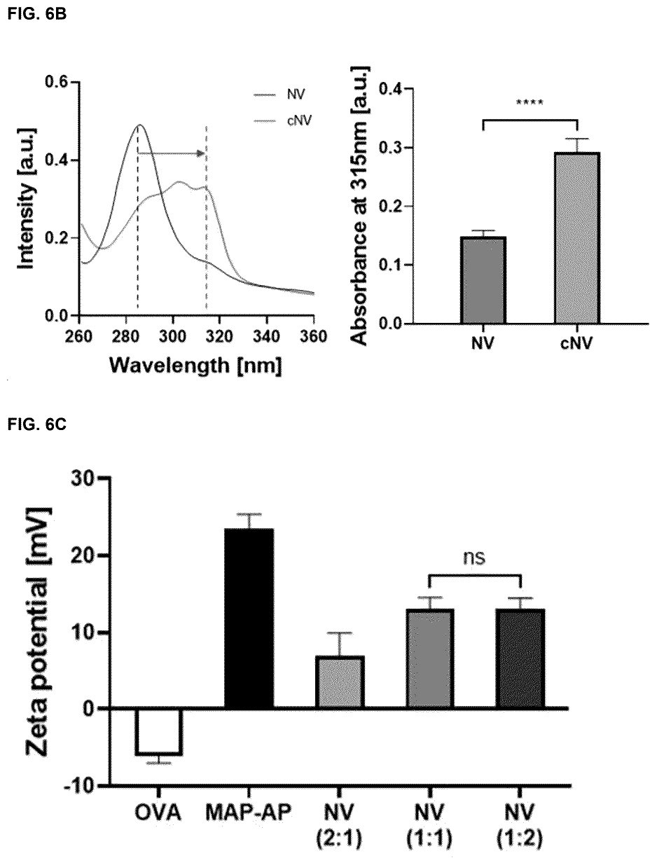

As a result, as shown in FIG. 6A, no significant changes in particle size and surface charge were observed between the nanocomplex (NV) before photoirradiation and the nanovaccine (cNV) after photoirradiation.

### 4-3. Analysis of Crosslinking Reactions Induced by Photoirradiation

Additionally, UV-Vis Spectroscopy was conducted to compare the spectra before and after photocrosslinking, confirming the occurrence of crosslinking.

First, the prepared nanocomplex before photoirradiation was diluted in D.W. to a concentration of 1 mg/ml, and 200 μl of the solution was placed into each well of a 96-well plate. The absorption spectrum was then measured in the range of 260 nm to 350 nm. Subsequently, blue light with a wavelength range of 450-470 nm was irradiated for 10 seconds, followed by measurement of the absorption spectrum in the same wavelength range.

As a result, as shown in FIG. 6B, a peak shift was observed from the pre-crosslinking state (black) to the post-crosslinking state (red). This confirms that the crosslinking was successfully induced by photoirradiation.

### 4-4. Analysis of Surface Charge of Nanocomplex Based on Mass Ratio of MAP-AP to Antigen

The surface charge was measured to investigate the formation of the nanocomplex based on the mass ratio of the adhesive adjuvant protein (MAP-AP) to the model antigen ovalbumin.

First, ovalbumin and the adhesive adjuvant protein (MAP-AP) were prepared at a concentration of 1 mg/mL each, and nanocomplexes were then prepared with ovalbumin and MAP-AP at weight ratios of 2, 1, and 0.5, respectively. Dynamic light scattering (DLS, Zetasizer, UK) was used to measure the surface charge of the samples. The concentration of each substance was maintained below 1 mg/mL, and all measurements were performed at room temperature.

As a result, the spontaneous formation of nanocomplexes by the negatively charged ovalbumin and adhesive adjuvant protein (MAP-AP) was confirmed through changes in surface charge. When the mass ratio of ovalbumin to MAP-AP reached 1 or higher, the adhesive adjuvant protein (MAP-AP) became saturated, and the mass ratio was ultimately set to 1.

These results indicate that the nanocomplex (MAP-AP/OVA nanocomplex) formed by mixing the model antigen ovalbumin with the adhesive adjuvant protein (MAP-AP), as well as the photocrosslinked nanovaccine (MAP-AP/OVA nanovaccine) formed by adding a photocrosslinking compound to the nanocomplex were successfully produced.

### Example 5: Freeze-Drying of MAP-AP-Based Nanocomplex (NV) and Nanovaccine (cNV)

### 5-1. Analysis of Surface Morphology and Structure Following Freeze-Drying

The surface morphology and structure of the nanocomplexes before and after freeze-drying (FD) were observed through scanning electron microscopy.

Specifically, the nanocomplex before freeze-drying and the nanocomplex after 24 hours of freeze-drying were each diluted in 10 ml of D.W., and 10 μl of the solution was placed on carbon tape, followed by an additional 12 hours of freeze-drying. Subsequently, the nanocomplexes before and after freeze-drying were gold-sputtered, and their particle morphology and structure were observed through scanning electron microscopy at accelerating voltages of 5 kV and 20 kV.

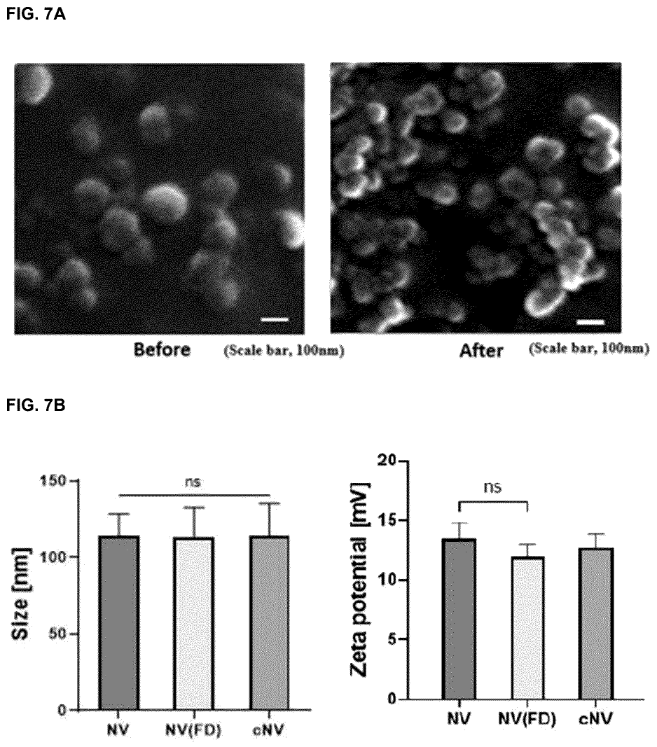

As a result, as shown in FIG. 7A, no changes in characteristics were observed after freeze-drying.

### 5-1. Analysis of Particle Size and Surface Charge Following Freeze-Drying

Additionally, an experiment was conducted to compare the particle size and surface charge of the nanocomplex (NV) before freeze-drying and the freeze-dried nanocomplex [(NV) (FD)] and nanovaccine (cNV).

Specifically, dynamic light scattering (DLS, Zetasizer, UK) was used to measure the particle size and surface charge, with each sample diluted in D.W. The particle concentration was maintained below 1 mg/mL, and all measurements were performed at room temperature.

As a result, as shown in FIG. 7B, no significant changes in particle size and surface charge were observed in the nanocomplex and nanovaccine before and after freeze-drying.

These results suggest the feasibility of long-term storage of nanocomplexes through freeze-drying.

### Example 6: Analysis of Antigen Release Profile of MAP-AP-Based Nanocomplex (NV) and Nanovaccine (cNV)

### 6-1. Analysis of Antigen Release Profile of Nanovaccine

An experiment was conducted to analyze the in vitro release profile of the nanocomplex and nanovaccine.

First, a 1 ml solution containing the nanocomplex prepared by the above method was placed in a dialysis membrane (MWCO 5000) containing 9 ml of PBS, and the tube was incubated in 30 mL of buffer solution with shaking at 37° C. At predetermined time intervals, 100 μl of each solution was sampled and replaced with fresh buffer solution. The amount of released ovalbumin was measured through ELISA.

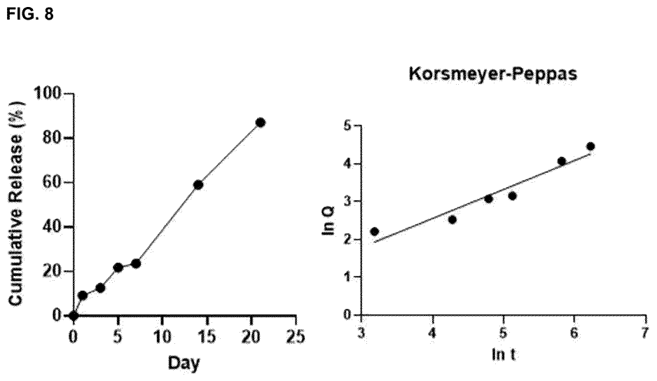

As a result, as shown in FIG. 8, ovalbumin was continuously released from the nanovaccine for a total of 21 days. Additionally, according to the Korsmeyer-Peppas analysis, 100% passive release was achieved in 23 days.

### 6-2. Analysis of Adhesion Ability of Nanovaccine

In addition, an experiment was conducted to analyze the adhesion ability of the nanovaccine.

Specifically, the adhesion of the particles was evaluated using a quartz crystal microbalance (QCM) device, with ovalbumin (OA) containing an Alum adjuvant as the control group. The Alum-adjuvanted group was prepared in the same manner as the aforementioned nanocomplex formation, and the prepared control group and nanovaccine were placed on a quartz crystal and dried for 1 hour. Subsequently, the weight was measured using the QCM device, followed by a 24-hour washing process in a PBS solution. The residual weight was then measured and compared.

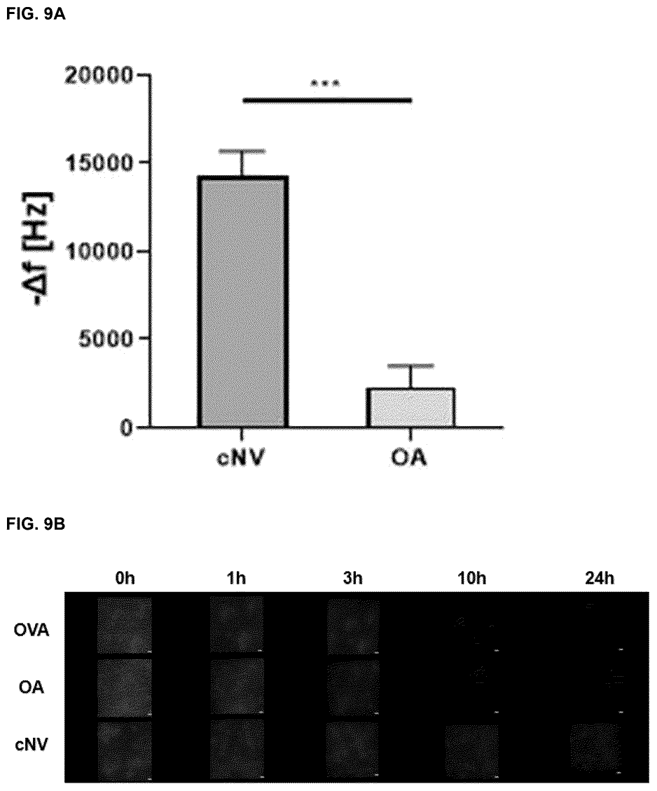

As a result, as shown in FIG. 9A, the nanovaccine (cNV) exhibited superior adhesion compared to the control group (OA).

### 6-3. Analysis of Tissue Adhesion Ability of Nanovaccines

Additionally, an experiment was conducted to confirm the tissue adhesion ability of the nanovaccines.

Specifically, the fluorescence of ovalbumin (OVA) containing a fluorescent dye (Texas-Red), ovalbumin (OA) with an Alum adjuvant, and ovalbumin with the nanovaccine (cNV), all distributed on pig skin tissue, was measured under a microscope.

As a result, as shown in FIG. 9B, the nanovaccine (cNV) exhibited superior tissue adhesion ability compared to the control group (OA) containing ovalbumin and the Alum adjuvant.

These results indicate that the nanovaccine of the present invention can continuously and locally release the ovalbumin antigen more efficiently than conventional Alum-based vaccines, owing to the adhesion ability of the adhesive adjuvant protein (MAP-AP).

### Example 7: Cytotoxicity and Biocompatibility Analysis of MAP-AP-Based Nanocomplex (NV) and Nanovaccine (cNV)

### 7-1. Cytotoxicity Analysis of Nanovaccine

To evaluate the cytotoxicity of the nanovaccine, a CCK-8 assay was performed after inoculating NIH3T3 fibroblast cells with the nanovaccine.

Specifically, 100 μg of the control group (OA), ovalbumin (OVA), nanocomplex (NV), untreated group (NC), 10% DMSO, and nanovaccine (cNV) were each applied to a monolayer of NIH3T3 cells, followed by CCK-8 assays at intervals of 24, 48, and 72 hours. The following evaluation experiment was conducted based on cell culture using the mouse fibroblast NIH3T3 cell line. NIH3T3 cells were cultured and maintained in DMEM containing 10% fetal bovine serum (FBS; Lonza) and 1% penicillin/streptomycin (Hyclone) under conditions of 5% CO₂ and 95% humidity at 37° C. The adherent cells were detached using 0.25% trypsin-EDTA, and viable cells, counted by the trypan blue assay, were used for further analysis. For toxicity evaluation, cells were initially seeded at 10⁵ cells per well in a 96-well culture plate. Each of the control group (OA), ovalbumin (OVA), nanocomplex (NV), untreated group (NC), 10% DMSO, and nanovaccine (cNV) was treated with 100 μg in the medium and cultured for 24, 48, and 72 hours, followed by cell viability measurement. Cell viability was determined by collecting 100 μl of culture medium, treating it with CCK-8 reagent (Dojindo, Japan), and culturing for 2 hours. The absorbance at 450 nm was then measured based on the aliquots from each medium.

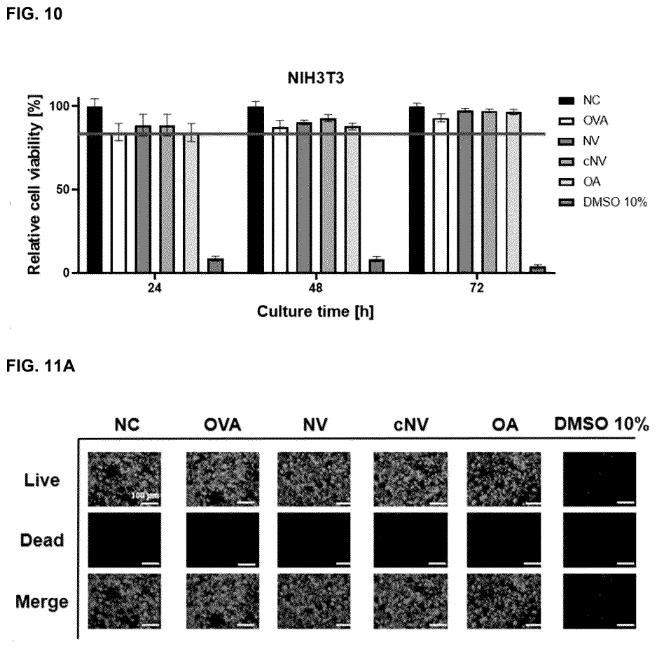

As a result, as shown in FIG. 10, minimal cytotoxicity was observed.

### 7-2. Biocompatibility Analysis of Nanovaccine

A Live/Dead assay was performed to evaluate the biocompatibility of the nanovaccine.

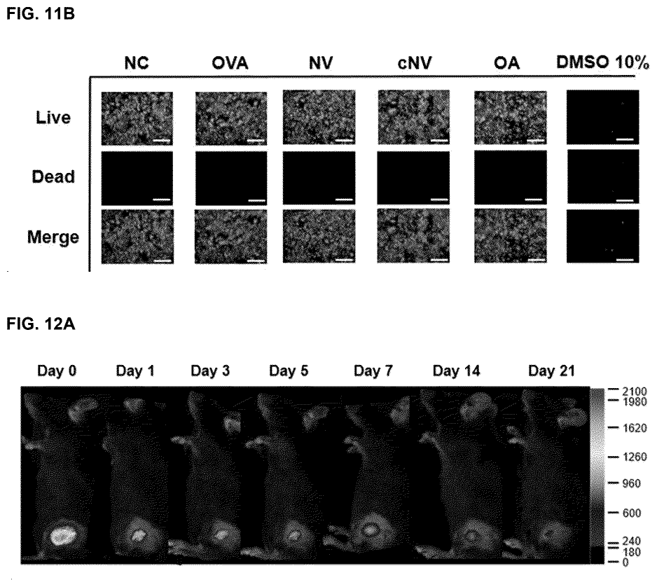

Specifically, 100 μg of the control group (OA), ovalbumin (OVA), nanocomplex (NV), untreated group (NC), 10% DMSO, and nanovaccine (cNV) were each applied to a monolayer of NIH3T3 fibroblast cells, followed by assays at intervals of 24 and 72 hours. The following evaluation experiment was conducted based on cell culture using the NIH3T3 cell line. NIH3T3 cells were cultured and maintained in DMEM containing 10% fetal bovine serum (FBS; Lonza) and 1% penicillin/streptomycin (Hyclone) under conditions of 5% CO₂ and 95% humidity at 37° C. The adherent cells were detached using 0.25% trypsin-EDTA, and viable cells, counted by the trypan blue assay, were used for further analysis. For toxicity evaluation, cells were initially seeded at 10⁴ cells per well in a 24-well culture plate. Each of the control group (OA), ovalbumin (OVA), nanocomplex (NV), untreated group (NC), 10% DMSO, and nanovaccine (cNV) was treated and cultured for 24 hours (FIG. 11A) and 72 hours (FIG. 11B), followed by Live/Dead assay.

As a result, as shown in FIGS. 11A and 11B, the nanocomplex (NV) containing the adhesive adjuvant protein (MAP-AP) and the photocrosslinkable compound, as well as the crosslinked nanovaccine (cNV), exhibited excellent cell viability with no significant differences compared to the other experimental groups, confirming that the biocompatibility of the adhesive adjuvant protein (MAP-AP) and the nanovaccine is as excellent as that of other adjuvants and antigens.

These results indicate that the nanovaccine according to the present invention exhibits low cytotoxicity, making it suitable for antigen delivery.

### Example 8: Analysis of In Vivo Persistence of MAP-AP-Based Nanocomplex (NV) and Nanovaccine (cNV)

An experiment was conducted to confirm the sustained in vivo delivery ability of the nanovaccine (cNV) of the present invention after subcutaneous inoculation into experimental mice.

Specifically, ovalbumin containing the fluorescent dye (Texas-Red) was used. The nanocomplex (NV) of the present invention, ovalbumin (OVA), and OVA/Alum (OA) were prepared with a 100 μg dose of ovalbumin each. Subsequently, the preparations were subcutaneously injected into 5-6 week-old C57BL/6 experimental mice. For the nanocomplex, blue light was irradiated for 10 seconds immediately after injection to induce in situ crosslinking, forming the nanovaccine (cNV). The in vivo behavior of ovalbumin was then monitored using an in vivo fluorescence imaging system on the day of injection, as well as on days 1, 3, 5, 7, 14, and 21.

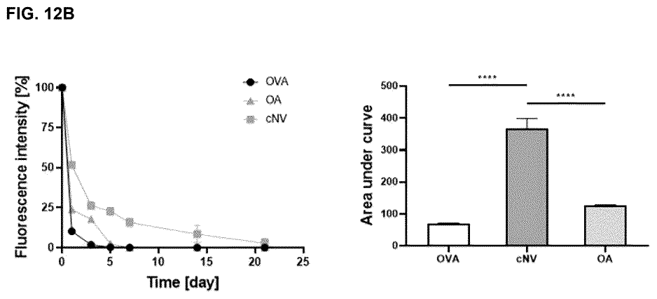

As a result, as shown in FIGS. 12A and 12B, unlike the OVA and OA groups, where the fluorescence disappeared after one week, the nanovaccine (cNV) group still exhibited fluorescence at the injection site even after two weeks.

These results indicate that the MAP-AP-based nanovaccine is locally maintained for an extended period in vivo, allowing for continuous antigen delivery over a relatively long duration.

### Example 9: Immune-Enhancing Effect of MAP-AP-Based Nanovaccine

The inventors of the present invention confirmed the efficacy of the nanovaccine, using the MAP-AP-based nanocomplex produced in Example 3, as an immune adjuvant.

### 9-1. Analysis of Antibody and Cytokine Concentrations in Serum

After vaccination in mice, blood and spleens were collected at intervals of 2 and 4 weeks to analyze the concentrations of OVA-specific antibodies (IgG, IgG1, and IgG2c) and cytokines (IL-4, IL-12, and IFN-γ) in each group. For blood analysis, plasma was separated from blood samples collected at 2 and 4 weeks post-vaccination, and the concentrations were verified through ELISA for ovalbumin-specific antibody analysis and ELISA for cytokine detection.

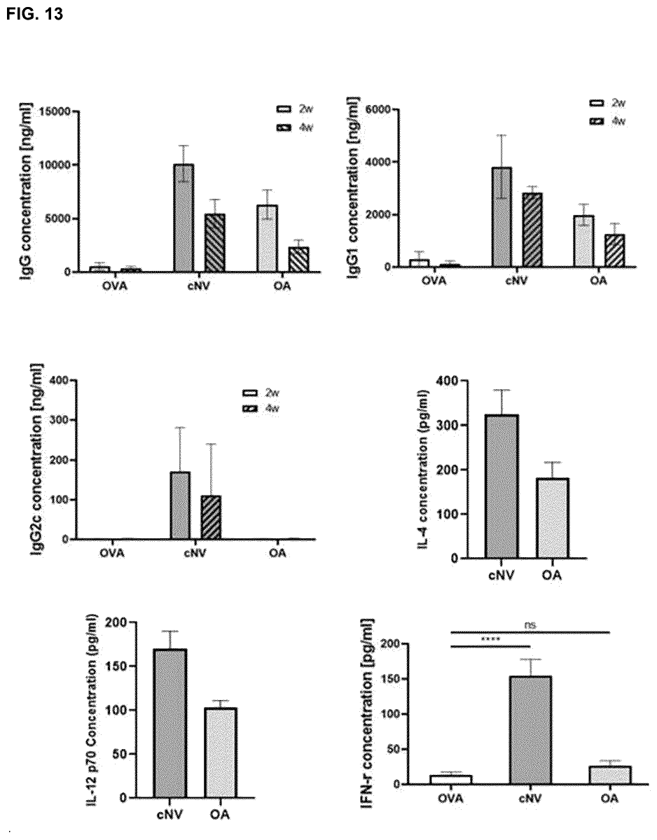

As a result, as shown in FIG. 13, even 4 weeks after the nanovaccine was injected into mice, antigen-specific antibodies and cytokines remained at high levels. Compared to OVA alone or OVA/Alum (OA), the nanovaccine exhibited significantly enhanced immune cell activation and immune response. Notably, compared to the Alum adjuvant, which is biased toward the Th2 immune response, the nanovaccine of the present invention maintained consistently high levels of not only IgG1, which is associated with the Th2 response, but also IgG2c, which is associated with the Th1 response. Additionally, cytokine analysis of blood samples collected after 2 weeks revealed that the nanovaccine exhibited higher IL-12 levels, indicating dendritic cell activation, compared to the Alum adjuvant. Simultaneously, compared to the Alum adjuvant and the OVA-alone group, which are biased toward the Th2 immune response, the nanovaccine of the present invention exhibited a higher concentration of IFN-γ, a cytokine associated with the Th1 immune response. Furthermore, the concentration of IL-4, a cytokine associated with the Th2 immune response, was also detected at higher levels compared to the Alum adjuvant.

These results are interpreted as being achieved due to the excellent antigen delivery and immune cell activation functions of the adhesive adjuvant protein.

### 9-2. Analysis of Immune Cell Activation in Splenocytes

A fluorescence-activated cell sorting (FACS) experiment was conducted to analyze the activation levels of immune responses in T-lymphocytes and dendritic cells among splenocytes.

Specifically, spleens collected 4 weeks post-inoculation were processed through a cell strainer to isolate cells. The isolated cells were then suspended in 1 ml of ACK lysis buffer and centrifuged at 2,000 RPM for 5 minutes to obtain splenocytes. Subsequently, the splenocytes were cultured in RPMI 1640 medium. The complete RPMI 1640 medium contained 10% FBS, 1% Penicillin, 1% L-Glutamine, and 1% HEPES buffer. Before the FACS experiment, the FACS buffer was prepared by adding 10% fetal bovine serum (FBS) to a DPBS solution. The splenocytes, which had been centrifuged at 2,000 RPM for 5 minutes, were then evenly resuspended in the FACS buffer. Subsequently, fluorescently labeled OVA was added and incubated in the dark for 10 minutes to facilitate its binding to immune cells, followed by three washes with FACS buffer. Next, mouse anti-CD4, anti-CD8a, anti-CD80, and anti-CD86 antibodies were added to the FACS buffer and then introduced to the splenocytes. The mixture was incubated in the dark for 10 minutes to induce antigen-antibody binding. After three additional washing steps, flow cytometry was performed to verify immune cells specifically activated by ovalbumin.

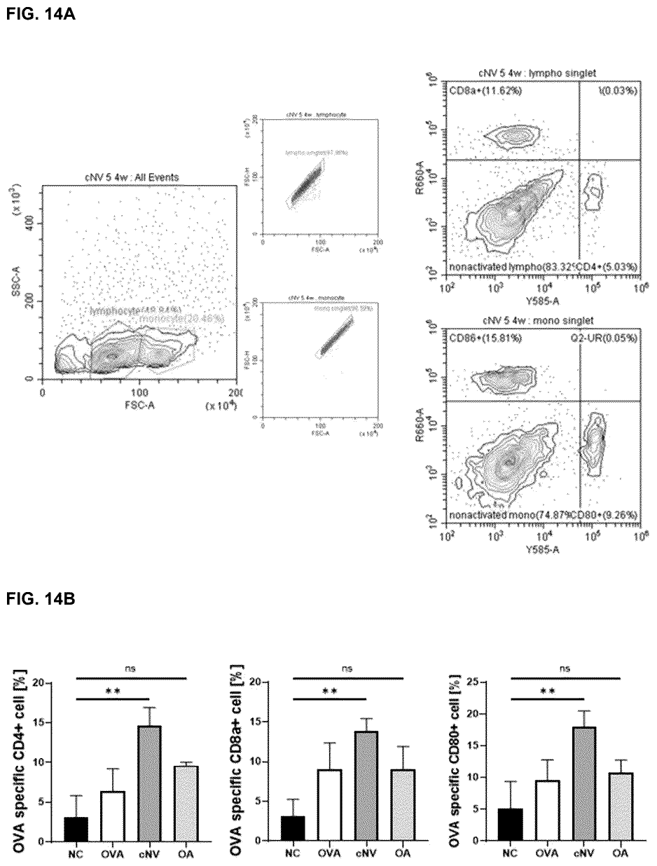

As a result, as shown in FIGS. 14A and 14B, the nanovaccine (cNV) of the present invention exhibited strong and sustained activation of splenic immune cells against ovalbumin even after 4 weeks post-injection. In particular, compared to OVA alone or OVA/Alum (OA), the nanovaccine demonstrated significantly higher activation of dendritic cells and Th1 immune cells, while also maintaining a high level of Th2 immune cell activation.

These results indicate that the nanovaccine based on the adhesive adjuvant protein of the present invention exhibits strong immune-enhancing effects and sustained antigen delivery capability. By mimicking natural infection, it not only induces robust activation of immune cells in vivo but also overcomes the limitations of conventional antigens and adjuvants by simultaneously activating the Th1 immune response. This, in turn, sustains high levels of antibodies and cytokines, ultimately eliciting a strong and long-lasting immune response.

In summary, the present invention achieves excellent vaccine delivery and immune-enhancing effects by developing a vaccine delivery carrier based on an adhesive adjuvant protein derived from mussel adhesive protein and producing a vaccine by incorporating an antigen through photocrosslinking treatment, making it effectively and safely applicable in the vaccine-related field.

What is claimed is:

1. A fusion protein in which an adjuvant sequence is fused to a mussel adhesive protein.

2. The fusion protein according to claim 1, wherein the mussel adhesive protein comprises one or more amino acid sequences selected from the group consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, and SEQ ID NO: 16, or a mutant thereof.

3. The fusion protein according to claim 1, wherein the adjuvant sequence is a T-helper epitope.

4. The fusion protein according to claim 3, wherein the T-helper epitope comprises at least one selected from the group consisting of a Pan DR-binding epitope (PADRE), a T-helper epitope of tetanus toxoid peptide (TTP), and a T-helper epitope of influenza virus hemagglutinin.

5. A nanocomplex comprising the fusion protein of claim 1 bound to an antigen.

6. The nanocomplex according to claim 5, comprising a photocrosslinker.

7. The nanocomplex according to claim 6, wherein the photocrosslinker is a photoinitiator or a photocatalyst.

8. The nanocomplex according to claim 7, wherein the photoinitiator comprises at least one selected from the group consisting of ruthenium (Ru(II)), palladium (Pd(II)), copper (Cu(II)), nickel (Ni(II)), manganese (Mn(II)), and iron (Fe(III)).

9. The nanocomplex according to claim 7, wherein the photocatalyst comprises at least one selected from the group consisting of persulfate, periodate, perbromate, perchlorate, vitamin B12, pentaamminechlorocobalt(III), ammonium cerium(IV) nitrate, oxalic acid, and EDTA.

10. A method for inducing an immune response in a subject, the method comprising administering the nanocomplex of claim 5 and a pharmaceutically acceptable carrier to the subject.

11. The method according to claim 10, wherein the method further comprising irradiating the nanocomplex with visible light.

12. The method according to claim 10, wherein the visible light induces cross-linking of a fusion protein in which an adjuvant sequence is fused to a mussel adhesive protein.

13. The method according to claim 10, wherein the method induces cellular and humoral immunity.
